(19) United States
(12) Patent Application Publication
(10) Pub. No.: US 2026/0000533 A1
(43) Pub. Date: Jan. 1, 2026

(54) DEVICES, SYSTEMS, AND METHODS FOR PATIENT POSITIONING AND MAINTAINING UPPER AIRWAY PATENCY

(71) Applicant: University of Pittsburgh - Of the Commonwealth System of Higher Education, Pittsburgh, PA (US)

(72) Inventors: Kirill Igorevich Kiselyov, Pittsburgh, PA (US); Ata Murat Kaynar, Pittsburgh, PA (US); Alyssa Zito, Pittsburgh, PA (US); Amelia Dubendorf Gordon, Berwyn, PA (US); Brandon Joseph Barber, Pittsburgh, PA (US); Kara Nghiem, Easton, PA (US)

(21) Appl. No.: 18/879,640

(22) PCT Filed: Jun. 28, 2023
(86) PCT No.: PCT/US2023/069270

§ 371 Date: Dec. 27, 2024

Related U.S. Application Data
(60) Provisional application No. 63/356,643, filed on Jun. 29, 2022.

(51) Int. Cl. A61F 5/56; A61B 5/00; A61B 5/0205; A61B 5/024; A61B 5/087; A61B 5/145

(52) U.S. Cl. CPC A61F 5/56; A61B 5/0205; A61B 5/024; A61B 5/087; A61B 5/14542; A61B 5/4818; A61B 2562/0204; A61B 2562/0219

(57) ABSTRACT

A system includes a positioning device including one or more movable contact members to contact the patient. one or more actuators in connection with the one or more movable contact members, and a support to which the actuators and the movable contact members are operatively connected. The support is configured or adapted to be placed in operative connection with the patient. The system further includes at least one source of en ergy in connection with the actuators and a control system in operative connection with the actuators and with the source of energy to control a state of the actuators via control of energy provided thereto to control a position of the movable contact members, and thereby to control a position of the patient in a determined manner.

### CROSS-REFERENCE TO RELATED APPLICATIONS

This application claims benefit of U.S. Provisional Patent Application Ser. No. 63/356,643, filed Jun. 29, 2022, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited berein are incorporated by reference.

Sleep-disordered breathing (for example, snoring and obstructive sleep apnea (OSA)) results from an interruption of airflow through the upper airway caused by airway obstruction by the upper palate, uvula, the tongue, or soft tissues and muscles surrounding the upper airway. Sleep-disordered breathing is a significant source of morbidity and mortality arising from the effects of interrupted breathing and sleep on mood, cognitive function, memory and cardiovascular function. Negative outcomes are especially pronounced in obese people, in aged people, and in people with upper respiratory infections (which may be transient) or other forms of upper airway obstruction. Anesthetics and the use of narcotic pain killers may significantly exacerbate the effects of OSA leading to potentially dangerous situations.

Common approaches to treating sleep-disordered breathing include continuous positive airway pressure (CPAP) devices, oral appliances and surgical interventions to either open the upper airways or stimulate the nerves and muscles leading to patency of upper airways. However, such approaches have many associated limitations. All such approaches are relatively expensive and invasive. Further, limitations are exacerbated in the hospital settings or in patients with recent traumas or disabilities. CPAP acceptance and compliance are not high and much worse among minorities. The effectiveness of CPAP declines in patients whose airways are congested as a result of upper respiratory infections. Oral appliances may be uncomfortable or impractical in patients with dentures, recent oral surgeries or trauma, or in emergencies. Similarly, surgical interventions to snoring are permanent, invasive, costly, and impractical in an emergency setting.

Similarly, sedation during or after procedures (for example, colonoscopy, or other minor surgical procedures) may result in an interruption of airflow through the upper airway caused by airway obstruction by the upper palate, uvula, the tongue, or soft tissues and muscles surrounding the upper airway unless acted on by the anesthesiologists or other health care providers. Common approaches to address anesthetic-induced airway obstruction requires vigilance by the anesthesiologists or health care providers towards maintenance of the airway patency.

Head tilt/chin lift (for example, the sniffing position, which is defined by neck flexion with upper cervical extension) is a non-surgical maneuver aimed at reducing airway obstruction by the collapsed upper palate or the tongue towards maintenance of the airway patency. Elements of the head tilt/chin lift approach (that is, head elevation) are present in certain pillow devices asserted to reduce sleep apnea and/or snoring. However, such devices typically work only when the user is sleeping on their back. Moreover, such devices are not readily adjustable to the changing circumstances of specific patients.

It remains desirable to develop devices, systems, and methods to provide improved results in addressing sleep-disordered breathing.

### SUMMARY

In one aspect, a system for use in connection with a patient includes a positioning device to control the position of a patient (and, particularly, the patient's head, chin, and neck—for example, during sleep). The head, chin, and neck may, for example, be positioned in a determined manner to increase comfort and/or to maintain upper airway patency (to a determined or desired level). The devices hereof may, for example, be used in treating patients experiencing sleep disordered breathing, in treating patients experiencing pulmonary hypertension, in periprocedural care, in treating patients experiencing anesthesia-induced airways obstruction, etc. The devices, systems, and methods hereof may also be used in healthy individuals to position that patient (and, particularly, the patient's head, chin, and neck) to, for example, increase comfort and/or maintain upper airway patency.

In another aspect, a system for use in connection with a patient includes a positioning device including one or more movable contact members to contact the patient, one or more actuators in connection with the one or more movable contact members, and a support to which each of the one or more actuators and each of the one or more contact members are operatively connected. The support is configured or adapted to be placed in operatively connection with the patient (for example, with at least a portion of the patient's neck). The system further includes at least one source of energy in connection with the one or more actuators and a control system in operative connection with the one or more actuators and with the at least one source of energy. The control system controls a state of the one or more actuators via control of energy provided thereto to control a position of the one or more movable contact members, and to thereby control a position of the patient in a determined manner. Control of the position of the patient may include control of the position of at least one of the patient's head, chin, or neck in a determined manner. The position of the patient's head, chin, or neck may, for example, be controlled to maintain upper airway patency. In a number of embodiments, the control system includes a processor system, a memory system in operative connection with the processor system, and at least one algorithm stored in the memory system and executable by the processor system to control the state of the one or more movable contact members. In a number of embodiments, at least part of the control system may be integrated within the positioning device. In a number of embodiments, the at least one source of energy includes a source of electrical energy, a source of hydraulic energy, or a source of pneumatic energy.

The system may further include a sensor system in operative connection with the control system. In a number of embodiments, the control system is configured to control the state at least one of the one or more actuators at least in part on the basis of data from a sensor system comprising one or more sensors. The sensor system may, for example, include at least one of a sound sensor, an oximeter sensor, a carbon dioxide sensor, a position sensor, a breath flow sensor, accelerometer, inclinometer, tilt sensor, a heart rate sensor, an ambient environment sensor (for example, temperature, humidity etc.), etc.

In a number of embodiments, the sensor system includes a sound sensor and the data from the sensor system includes data on the sound of the patient's breathing. The control system may be configured to determine at least one of a state of the patient's breathing and a position of the patient from the data from the sensor system and control the state of the one or more actuators at least in part on the basis of at least one of the determined state of the patient's breathing and the position of the patient.

In a number of embodiments, the at least one algorithm is configured to store a maximum state and a minimum state for at least one of the one or more actuators. The maximum state and the minimum state are determined via patient interaction.

The at least one algorithm may be configured to implement feedback control of the state of the at least one of the one or more actuators on the basis of data from the sensor system. In a number of embodiments, the control system includes at least one machine learning algorithm stored in the memory system and executable by the processor system to alter control of the state of the at least one of the one or more actuators on the basis of data from the sensor system measured over time. The sensor system may include a sound sensor and the machine learning algorithm is configured to differentiate snoring (and/or other breathing conditions) from other sounds. The machine learning algorithm may, for example, be configured to differentiate between sounds coming from more than one person. The machine learning algorithm may be continuously trained or improved based on data received over time from the sensor system.

At least one of the one or more contact members is positioned to be placed in contact with a lower portion of the jaw line of the patient in a number of embodiments. At least one of the one or more contact members may be positioned to be placed in contact with the neck of the patient.

In a number of embodiments, the support includes a frame comprising a lower section configured to contact a chest region of the patient and an upper section includes one or more of the one or more movable contact members. The upper section is movable relative to the lower section. At least one of the one or more actuators is placed in connection with the upper section and with the lower section to move the upper section relative to the lower section. In a number of embodiments, the upper section is connected to the lower section by a first resilient member on a first side of the frame and by a second resilient member on a second side of the frame. The upper section and the lower section may be removably connected to the first resilient member and the second resilient member and the first resilient member and the second resilient member are selectable for the patient. In a number of embodiments, the system includes a first actuator on the first side of the frame which is in connection with the upper section and with the lower section and a second actuator on the second side of the frame which is in connection with the upper section and with the lower section. The control system may be configured to independently control the first actuator and the second actuator and thereby independently control the amount of flex in each of the first resilient member and the second resilient member.

In a number of embodiment of systems hereof, the source of energy includes a pneumatic source of energy, and at least one of the one or more actuators includes an inflatable system in fluid connection with the pneumatic source of energy. The state of the at least one of the one or more actuators which is controlled by the control system is a state of inflation. Each of the one or more actuators may include an inflatable system in fluid connection with the pneumatic source of energy and the state each of the one or more actuators controlled by the control system may be a state of inflation. The control system may be configured to control the state of inflation of at least one of the inflatable systems independently of the control of at least one other of the plurality of inflatable systems.

In a number of embodiments, the inflatable system includes an inflatable chamber formed at least partially of a flexible material (for example, a generally gas impermeable, flexible material). The inflatable system may include a plurality of connected inflatable chambers, wherein each of the plurality of connected inflatable chambers is formed at least partially of a flexible material. Each of the plurality of connected inflatable chambers may be in fluid connection with the others of the plurality of inflatable chambers and pneumatic energy may be provided to the inflatable system via a single conduit.

In a number of embodiments, the none of the plurality of connected inflatable chambers is in fluid connection with the others of the plurality of inflatable chambers and pneumatic energy is provided to each of the plurality of connected inflatable chambers by a separate conduit. The pneumatic energy provided to each of the plurality of inflatable chambers is independently controllable via the control system. Two or more of the plurality of connected inflatable chambers may be connected in a manner to control inflation of the inflatable system.

In a number of embodiments, the inflatable system is formed at least partially of a flexible material and at least a portion the flexible material forms at least a portion of one of the one or more movable contact member. In that regards, an actuator such as an inflatable system here may include or form at least a portion of a movable contact member hereof. In a number of embodiments including a plurality of actuators, each of the plurality of actuators includes an inflatable system which is formed at least partially of a flexible material. At least a portion of the flexible material of two or more of the plurality of actuators forms at least a portion of two or more of the movable contact members. In a number of embodiments, two or more of the plurality of actuators form at least a portion of the support.

Two or more of the plurality of actuators may be positioned at different circumferential positions on the support, wherein the support extends around at least a portion of the circumference of the patient's neck when the positioning device is placed in operative connection with the patient's neck.

In another aspect, positioning device for use in connection with a patient, includes one or more movable contact members to contact the patient, one or more actuators in connection with the one or more movable contact members, and a support to which each of the one or more actuators and each of the one or more contact members are operatively connected. The support is configured to be placed in operative connection with the patient (for example, with at least a portion of the patient's neck). Each of the one or more actuators is adapted of configured to be placed in connection with at least one source energy. A state of the one or more actuators is controllable via control of energy provided thereto from the at least one source of energy to control a position of the one or more movable contact members and thereby control a position of the patient in a determined manner. Control of the position of the patient may include control of the position of at least one of the patient's head, chin, or neck in a determined manner. The position of the patient's head, chin, or neck may, for example, be controlled to maintain upper airway patency. The positioning device may be further characterized as described above and elsewhere herein.

In another aspect, a method of positioning a patient includes providing a system which includes a positioning device including one or more movable contact members to contact the patient, one or more actuators in connection with the one or more movable contact members, and a support to which each of the one or more actuators and each of the one or more contact members are operatively connected. The support is configured or adapted to be placed in operatively connection with the patient (for example, with at least a portion of the patient's neck). The system further includes at least one source of energy in connection with the one or more actuators and a control system in operative connection with the one or more actuators and with the at least one source of energy. The control system controls a state of the one or more actuators via control of energy provided thereto to control a position of the one or more movable contact members, and thereby to control a position of the patient in a determined manner. Control of the position of the patient may include control of the position of at least one of the patient's head, chin, or neck in a determined manner. The position of the patient's head, chin, or neck may, for example, be controlled to maintain upper airway patency. In a number of embodiments, the control system includes a processor system, a memory system in operative connection with the processor system, and at least one algorithm stored in the memory system and executable by the processor system to control the state of the one or more movable contact members. In a number of embodiments, at least part of the control system may be integrated within the positioning device. In a number of embodiments, the at least one source of energy includes a source of electrical energy, a source of hydraulic energy, or a source of pneumatic energy. The method further includes placing the support in operative connection with the patient and operating the control system to control a position of the one or more movable contact members and thereby control a position of the patient's head in the determined manner. The method may be further characterized as described above and elsewhere herein.

In a further aspect, a method of positioning a patient, includes (i) providing a positioning device which includes one or more movable contact members to contact the patient, one or more actuators in connection with the one or more movable contact members, and a support to which each of the one or more actuators and each of the one or more contact members are operatively connected, the support being configured to be placed in operative connection with the patient, each of the one or more actuators being adapted to be placed in connection with at least one source energy, wherein a state of the one or more actuators is controllable via control of energy provided thereto from the at least one source of energy to control a position of the oner or more movable contact members and thereby control a position the patient's head in a determined manner, and (ii) placing the positioning device in operative connection with the patient. The method may further include controlling a state of the one or more actuators by control of energy provided thereto from the at least one source of energy to control a position of the one or more movable contact members and thereby the position of the patient in the determined manner. Control of the position of the patient may include control of the position of at least one of the patient's head, chin, or neck in a determined manner. The position of the patient's head, chin, or neck may, for example, be controlled to maintain upper airway patency.

In still a further aspect, a system includes a device to maintain patency of upper airways of a patient and a control system in operative connection with the device. The control system includes a processor system, a memory system in operative connection with the processor system, and one or more algorithms stored in the memory system and executable by the processor system to control the device. The one or more algorithms includes at least one machine learning algorithm. The machine learning algorithm is configured to alter control of the device on the basis of data from a sensor system measured over time. In a number of embodiments, the sensor system includes a sound sensor and the machine learning algorithm is configured to differentiate snoring from other sounds. The machine learning algorithm may, in a number of embodiments, be configured to differentiate between sounds coming from more than one person. In a number of embodiments, the device is a positioning device to, for example, control the position of at least one of the patient's head, chin, or neck in a determined manner to maintain patency of the upper airways of the patient.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a side view of a person in the supine position with the head, chin and neck positioned in a representative desirable position to, for example, reduce the effects of sleep-disordered breathing.

FIG. 2A illustrates schematically a perspective view of a representative embodiment of a system hereof including an adjustable positioning device.

FIG. 2B illustrates schematically an embodiment of a sleep sound/snoring detection and characterization device, system and method hereof.

FIG. 3A illustrates several views of another embodiment of an adjustable positioning device hereof positioned on the neck of a user.

FIG. 3B illustrates a flowchart of an embodiment of a control methodology for a system hereof including three expandable chambers.

FIG. 4 illustrates a perspective view of another embodiment of an adjustable positioning device hereof.

FIG. 5A illustrates schematically an embodiment of a two-chamber bellows system hereof in a disassembled state.

FIG. 5B illustrates schematically an assembled, two-chamber bellows system hereof in an each of a deflated (left) and inflated (right) state.

FIG. 5C illustrates schematically an assembled, three-chamber bellows system hereof in an each of a deflated (left) and inflated (right) state.

FIG. 5D illustrates a perspective view of an embodiment of a two-chamber bellows system hereof wherein the chambers may be adhered/connected on one side thereof to provide comparatively increased expansion on the other side.

FIG. 5E illustrates perspective view of an embodiment of a two-chamber bellows system of FIG. 5D wherein the chambers are adhered/connected on one side thereof to allow controlled expansion on the other side.

FIG. 5F illustrates a side view of an assembled, two-chamber bellows system hereof wherein each chamber has an independent air supply line and is attached to the adjacent chamber.

FIG. 5G illustrates a side view of an assembled, three-chamber bellows system or stacked chamber system hereof wherein each chamber has an independent air supply line and is attached to the adjacent chamber(s).

FIG. 5H illustrates a side view of an assembled, three-chamber bellows system hereof wherein each chamber has an independent air supply line and is attached to the adjacent chamber(s) on different axes of attachment.

FIG. 6A illustrates schematically a flow path for an embodiment of a system hereof including two stacked-chamber or bellows systems (a right-side or right bellows system and a left-side or left bellows system) and demonstrating air flow to the bellows system for inflation thereof and flow from the bellows system in the case of air release/deflation.

FIG. 6B illustrates schematically a flow path for another embodiment of a system hereof including two, three-chamber bellows systems (a right-side or right bellows system and a left-side or left bellows system).

FIG. 7A illustrates schematically a side view of another embodiment of an adjustable positioning device hereof in operative connection with a person in a supine position, wherein the device includes adjustable chambers or bellows positioned on a support including a frame, wherein the device is in an inactivated state.

FIG. 7B illustrates schematically a side view of the adjustable positioning device of FIG. 7A, wherein the device is in an activated state.

FIG. 8A illustrates schematically a side view of another embodiment of an adjustable positioning device hereof in operative connection with a person in a supine position, wherein the device includes an adjustable bellows system positioned in connection with a support including a frame which includes a lower or base section and an upper section which contact the user, wherein the bellows system is inflatable to control the position of the upper section of the frame which is adapted or operable to contact a person's chin/jaw or tissue at the base of the mandible, and wherein the device is in an inactivated state.

FIG. 8B illustrates schematically another side view of the device of FIG. 8A wherein the device has been placed in an activated state in which the bellows system has been expanded such that the upper section of the frame/support is moved to contact the person's chin/jaw to place the patient's head in the approximate orientation of FIG. 1.

FIG. 9 illustrates schematically an adjustable position device hereof including the bellows system of FIG. 5E incorporated within a frame of the adjustable position device, wherein the device is in operative connection with a person who is holding a hand-held control system for the adjustable positioning device.

FIG. 10A illustrates a side view of another embodiment of an adjustable positioning device hereof in operative connection with a person in a supine position, wherein the device includes two adjustable bellows systems positioned between a lower/base section and an upper section on a left side and a right side of a frame to control the position of the upper section, which is operable to contact a person's chin/jaw or tissue at the base of the mandible, and wherein the device is in an activated state.

FIG. 10B illustrates a top view of the device of FIG. 10A in operative connection with the person, wherein the device is in an activated state.

FIG. 11A illustrates a front view of another embodiment of an adjustable positioning device hereof wherein the device includes two adjustable bellows systems positioned between a lower/base section and an upper section on a right side and a left side of a frame.

FIG.11B illustrates a side view of the device of FIG. 11A.

FIG. 12A illustrates a perspective view of another embodiment of an adjustable positioning device hereof without the adjustable bellows systems attached thereto.

FIG.12B illustrates another perspective view of the device of FIG. 12A including a lower frame or base housing.

FIG. 12C illustrates a top plan, upper and bottom views of a section or portion of a frame of another embodiment of an adjustable positioning device hereof without adjustable bellows systems attached thereto.

FIG. 13 illustrates a generalized flow chart for operation of a device or system hereof.

FIG. 14A, in combination with FIGS. 14B and 14C, illustrates a more detailed flow chart for operation of a device or system hereof, wherein FIG. 14A illustrates an algorithm for the determination of sleep position and for control/operation after determination of a sleep on back position.

FIG. 14B illustrates a portion of the operation flow chart setting forth an algorithm for control after determination of a left-side sleep position.

FIG. 14C illustrates another portion of the operation flow chart setting forth an algorithm for control after a determination of a right-side sleeping position.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “an inflatable chamber” includes a plurality of such inflatable chambers and equivalents thereof known to those skilled in the art, and so forth, and reference to “the inflatable chamber” is a reference to one or more such inflatable chambers and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”, “circuitry” or “circuit,” as used herein include, but are not limited to, hardware, firmware, software, or combinations of each to perform a function(s) or an action(s). For example, based on a desired feature or need, a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor” or “processor system”, as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

As used herein, the term “personal communications device” refers to a portable or mobile device which includes a communication system, a processor system, a user interface system (for example, a visual feedback system including a touchscreen or other display, an auditory feedback system, and a tactile feedback system, a user input system etc.) and an operating system capable of running general-purpose applications. Examples of personal communications devices include, but are not limited to, smartphones, tablet computers and custom devices. As used herein, the term “tablet computer” or tablet, refers to a mobile computer with a communication system, a processor system, at least one user interface as described above (typically including a touchscreen display), and an operating system capable of running general-purpose applications in a single unit. As used herein, the term “smartphone” refers to a cellular telephone including a processor system, at least one user interface as described above (typically including a touchscreen display), and an operating system capable of running general-purpose applications. Such personal communication devices are typically powered by rechargeable batteries and are housed as a single, mobile unit. Moreover, in a number of embodiments personal communications devices are able accept input directly into a touchscreen (as opposed to requiring a keyboard and/or a mouse). Personal communications devices typically provide for internet access through cellular networks and/or wireless internet access points connected to routers. A number of representative embodiments of systems and/or methods hereof are discussed in connection with the user of a smartphone as the personal communication device.

In a number of representative embodiments, the devices, systems, and methods hereof provide improved results in addressing a condition such as sleep-disordered breathing/sleep apnea, pulmonary hypertension, conditions related to periprocedural care, conditions related to sedation during or after procedures, etc. Limitations of existing devices, system, and methods used in connection with such representative disorders are reduced or eliminated in a number of embodiments hereof through use of active positioning device or system to achieve a desired patient position (for example, a determined head position—for example, head tilt and chin lift). The devices, systems, and methods hereof may, for example, include a feedback mechanism such as a sensor-based feedback system which interacts with a control system to control the positioning devices, systems, and methods to adjust control based upon changing data regarding the patient received from the sensor system over time. The active positioning devices hereof can be used to address sleep-disorders and/or other conditions of a patient regardless of the patient's sleep position (for example, with the patient on the patient's back, on the patient's side, or on the patient's front/stomach.)

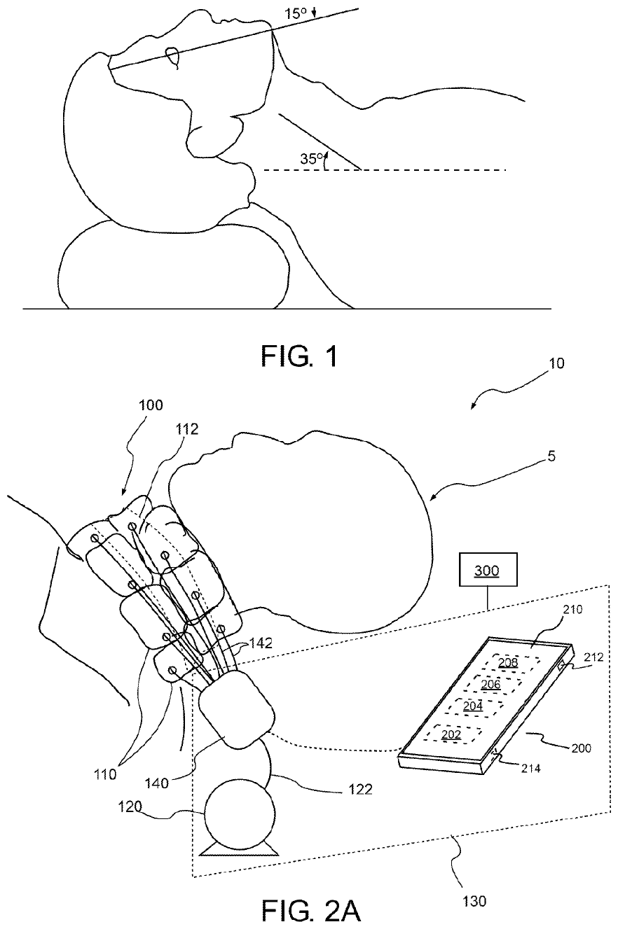

FIG. 1 illustrates a side view of a person in the supine position with the patient (for example, the patients' head, chin and neck) positioned in a representative desirable position to reduce the effects of, for example, sleep-disordered breathing by reducing obstruction of an individual's airway by the palate or tongue (that is, by maintaining suitable patency of the upper airways). The illustrated position is sometimes referred to as a “sniffing position” (used in, for example, the medical field) which may, for example, be achieved by placing a patient in supine position with a 10-cm pillow under the head to attain 15° face-plane extension and 35° neck flexion.

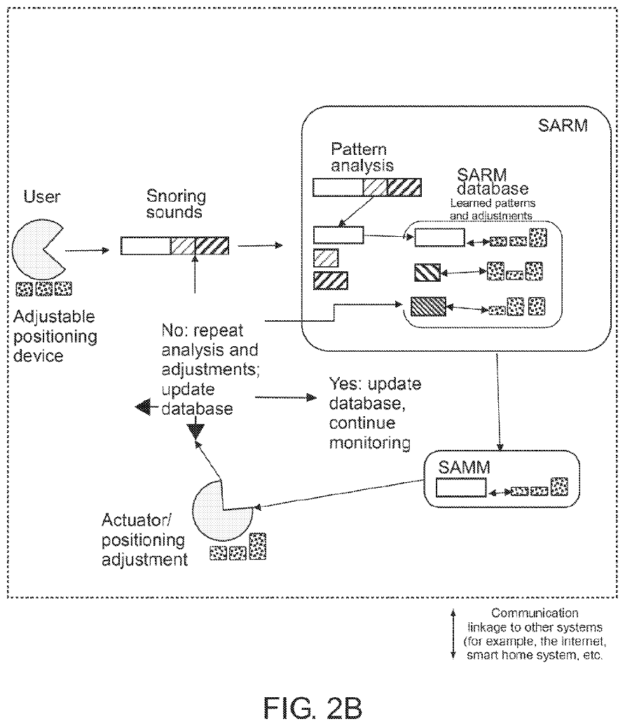

FIG. 2 illustrates a representative embodiment of a system 10 hereof for use with a patient 5 which includes a positioning device 100 to adjust neck angle, head tilt/angle and/or chin lift/angle (or the angle of the mandible) in positioning the patient's head to address sleep-disordered breathing. In general, positioning device 100 and other positioning devices hereof include one or more movable interface or contact members to interface with or contact the patient's head and/or neck to control the position of the patient's head to, for example, facilitate breathing/patency of the upper airways. One or more actuators are placed in connection with the one or more contact member to control the state thereof and thereby to control the position of the head via contact with the head and/or neck as described herein. As used herein, the term “actuator” refers to a component or element that achieves physical motion by converting energy (for example, electrical energy, hydraulic energy, or pneumatic energy) into mechanical force to perform a mechanical action or movement.

In the embodiment illustrated in FIG. 2, positioning device 100 includes a plurality of inflatable systems such as inflatable volumes, chambers, compartments, bladders or bellows 110 which may operate as actuators as well as moveable contact elements bereof. Other types of moveable member or actuators (for example, expandable/contractable actuators, extendable/retractable actuators, etc.) such as hydraulically operated members/actuator, and/or electromechanically operated members/actuators may be used. Representative embodiments of devices, systems, and/or methods hereof are discussed in connection with movable members/actuators including inflatable systems (for example, inflatable chambers and/or multi- or composite-chamber systems, sometimes referred to herein as bellows systems). Such inflatable systems are pneumatically energize/inflated (for example, via a source of pressurized air). Use of inflatable systems may for example, provide increased comfort (via, for example, a pillowing or dampening effect) and may, in certain circumstance provide increased safety as compared to other actuators.

In the embodiment illustrated in FIG. 2, inflatable chambers 110 are in fluid connection with a source of pressurized gas (typically air). Portions of the surface of chambers 110 function as interface or contact members to contact the patient and position the patient's head to a determined or desired position. Inflatable chambers 110 are maintained in connection with the patient's neck via a support or support system 112 illustrated schematically in FIG. 2. Support system 112 may, for example, include an adjustable, soft collar or strap to be donned upon and conform to the patient's neck. In the illustrated embodiment, the source of pressurized gas is an air pump 120. In the illustrated embodiment, air pump 120 is in fluid connection with a controllable manifold or distribution system 140 via a line or conduit 122. Air pump 120 is illustrated as a separate component in system 10 of FIG. 2, but it may be incorporated into positioning device 100. Moreover, a plurality of air pumps may be provided. Indeed, in a number of embodiments, separate air pumps may be provided for each inflatable chamber 110. Controllable manifold or distribution system 140 is in fluid connection with each of inflatable chambers 110 via lines or conduits 142. Controllable manifold or distribution system 140 may, for example, be part of a control system 130 that can include a controller/processor system. Electronic circuitry of system 10 may, for example, include control system 130 which may, for example, include a processor system, a memory system in operative connection with the processor system, a communication system, a user interface system, etc. The memory system may include one or more software algorithms stored therein and executable via the processor system to control system 10. Components of the control system 130 may, for example, be embodied in a dedicated controller and/or in a personal communication device such as smartphone 200 as illustrated in FIG. 2.

As illustrated in FIG. 2, in a number of embodiments, control system 130 includes a smartphone 200. As known in the art and as illustrated schematically in FIG. 2, smartphone 200, includes a processor system 202, a memory system 204, a communication system 206 (which may, for example, include wireless cellular telephone connectivity (providing telephone and internet connectivity), radio-band or Wi-Fi internet connectivity, BLUETOOTH wireless connectivity, infrared wireless connectivity, etc.) and an interface system 208 (including, for example, a touchscreen display 210, a microphone/speaker system 212, and a vibration mechanism). Smartphone 200 may also include a sensor system 214 including, for example, sound sensor etc. Such sensors may be incorporated into or form a part of sensor system 300 of the systems, devices and methods hereof, which is in operative connection with control system 130. Smartphone 200 may be incorporated into or form all or a part of one or more components of control system 130.

Each of inflatable chambers 110 is independently inflatable over a range of, for example, 0 to 100% to adjust head position (via, for example, adjustment of neck angle, head tilt and/or chin lift) in a determined manner via control system 130. Using feedback from the one or more sensors of sensor system 300 (for example, sound sensor(s), oximeter sensor(s), heart rate sensor(s), body temperature sensor(s), ambient temperature sensor(s), carbon dioxide sensor(s), IMU/position sensors(s), accelerometer(s), inclinometer(s), tilt sensor(s), breath flow sensor(s), pressure sensors, etc.) inflatable chamber 110 may be independently controlled/inflated to achieve a determined position which may change over time in response to patient and ambient parameters. Carbon dioxide may, for example, be a transcutaneous carbon dioxide sensor as disclosed in Transcutaneous CO₂ Monitoring, AAST Technical Guideline, American Association of Sleep Technologists (2018). Various sensors may be included from independent monitors. For example, a patient may be monitored in a procedure room with standard monitors, such as pulse oximeter and the data from such monitors/sensor may be communicated to an incorporated in the devices or systems hereof.

Accelerometer data may, for example, be used to determine upon which side the user is laying. Depending on the side on which the user is laying, the control system hereof may change which chamber(s) is/are inflated (for example, on the left side, the right side, or both) as well as the extent of inflation. Code hereof may, for example, incorporate a low pass filter to prevent small or quick movements made during sleep from triggering an inflation code/command. As clear to one skilled in the art, the manner in which the side upon which the user is laying will depend upon the placement of the accelerometer on the user's body.

A sound sensor or microphone may be used for sound detection and classification. Using, for example, a software algorithm such as a machine learning algorithm. With such software algorithms (for example, as available from Edge Impulse Inc. of San Jose, California and/or “Arduino” software), one may create a library that allows the control system to differentiate between snoring and other noises. Moreover, it may also be possible to differentiate, for example, between the snoring of different people.

FIG. 2B illustrates an embodiment of a snoring detection and characterization system and method hereof. In that regard, in a number of embodiments, a software algorithm hereof can includes a Snore Analysis and Response Module (SARM), which provides analysis, learning, and adjustment of subsystems. The SARM is a specialized software module based on real-time analysis and learning of, for example, sound/snoring patterns. The SARM uses such information (and/or other data) to initiate a sequence of corrective adjustments to the user's sleeping position via adjustment of the actuators (for example, inflatable chambers 110) of an adjustable positioning device hereof (for example, device 100.

A Sound Pattern Analysis Subsystem (SPAS) may, for example, performs real-time digital signal processing on the collected sound and/or other data. The subsystem may, for example, use machine learning algorithms to detect and learn patterns or phases in the snoring sounds, differentiating between ordinary noise and characteristic snoring sounds, and identifying specific combinations of frequencies and other characteristics of different patterns of snoring noise for individual patients. Such patterns are stored in an associated database (for example, an online database or a database incorporated in the electronic circuitry of device 100 or an adjacent device). In addition, the SPAS module may, for example, learn (using, for example, a Smart Adjustment Mechanism Subsystem or SAMS subsystem described further below) which head positions and combinations of actuator adjustments (for example, inflation patterns as represented schematically on FIG. 2B) are most likely to cause the cessation of specific noise patterns of an individual patient. Such information is updated (learned) during each sleep session. The information is stored in the SPAS database.

The Smart Adjustment Mechanism Subsystem or SAMS adjusts the sleeper's position based on the output from the SPAS. Such adjustment may, for example, involve inflating or deflating specific inflatable actuators within an adjustable positioning device hereof which, in certain embodiments, may operate or function as a specialized pillow to gently adjust the sleeper's head or neck position. In a representative embodiment, when the SPAS detects a specific pattern of snoring noise, it instructs the SAMS to initiate the corresponding optimal inflation protocol, using the database stored in SAMS. In parallel, SAMS learns the outcome of the head adjustment, updates the database, and initiates an adjustment if necessary.

In a number of representative embodiments, the device or system hereof begins operation once the user is ready to sleep. The sound detection module starts capturing sounds as soon as it is activated. Such raw audio data is then passed to the SARM, where it is processed and analyzed for, for example, sound frequencies and phases that signify specific patterns of snoring. Upon detection of such patterns, the SARM communicates with the SAMS. The type of adjustment is determined based on a specific snoring pattern detected. For example, if the pattern suggests obstruction in the nasal airways, the device may raise the sleeper's head slightly.

The device/system hereof may continually monitor the user's data (which may be associated with snoring), making adjustments as needed throughout the period of sleep. An objective is to alleviate snoring by dynamically responding to changes in the user's snoring patterns. The devices, systems, and methods hereof provide an innovative approach to managing snoring by adapting to the user's needs in real time. The devices, systems, and methods hereof not only have the potential to improve the quality of sleep for the user but also those in the vicinity of the user. In addition to user positioning, the devices, systems, and methods hereof may also control other conditions of the user's environment (for example, the room ambient temperature or humidity) through, for example, a communication linkage to a smart home system.

One or more pressure sensors may, for example, be used to detect when the chambers hereof are sufficiently filled. Data from such sensor may be used by the control system hereof to control the pump to prevent excessive air pressure into the chambers. Pressure sensor can also be used to inflate chambers partially to, for example, control the amount of chin tilt.

By, for example, independently inflating and deflating chambers 110 in response to the measured sound of sleep-disordered breathing, system 10 may automatically adjust the chin and head positioning to a first state until such sound (for example, snoring) subsides. System 10 may, for example, maintain the chin and head position in the first state unless and until the sound of sleep-disordered breathing resumes, which will initiate another round of adjustment. As described above, algorithms of control system 130 may, for example, include learning algorithms/artificial intelligence algorithms such as machine learning algorithms as known in the art to improve patient-specific and/or patient population adjustment outcomes based on data measured over time for one or more patients, ambient environmental data, etc. In a number of embodiments inflatable chambers 110 may be first inflated to a predetermined state (based, for example, upon one or more measured control variable such as positions of chambers 110, pressure in chambers 110, volume of chambers 110, neck orientation, body position, etc.) for a given patient. The predetermined state may, for example, be learned over repeated use cycles using, for example, one or more machine learning algorithms.

In a number of embodiments, a user may perform a setup or customization of a device or system hereof. For example, a user may manually or otherwise control inflation of one or more chambers of a system hereof until the user's chin is comfortably lifted to the “sniffing” position. That position may, for example, be saved as a max inflation setting (pressure and volume). A minimum inflated position can also be set to the user's preference. For example, the user may find a slightly higher chin level to be a comfortable baseline. The user may, for example, also place limits on side-to-side inflation based on comfort and sleeping habits. Timer settings may, for example, be incorporated for specific hours of operation. Alarm setting may, for example, be incorporated to wake the user at a specific time.

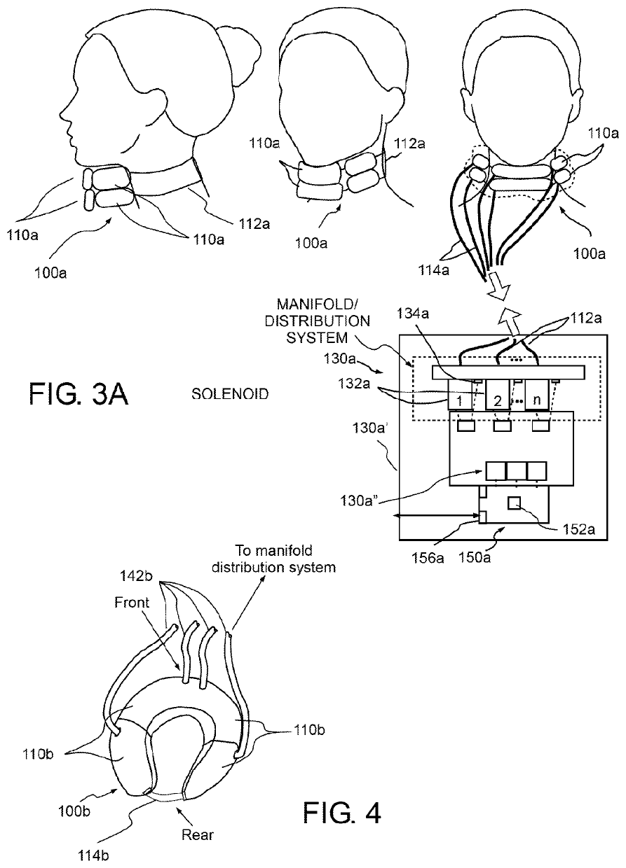

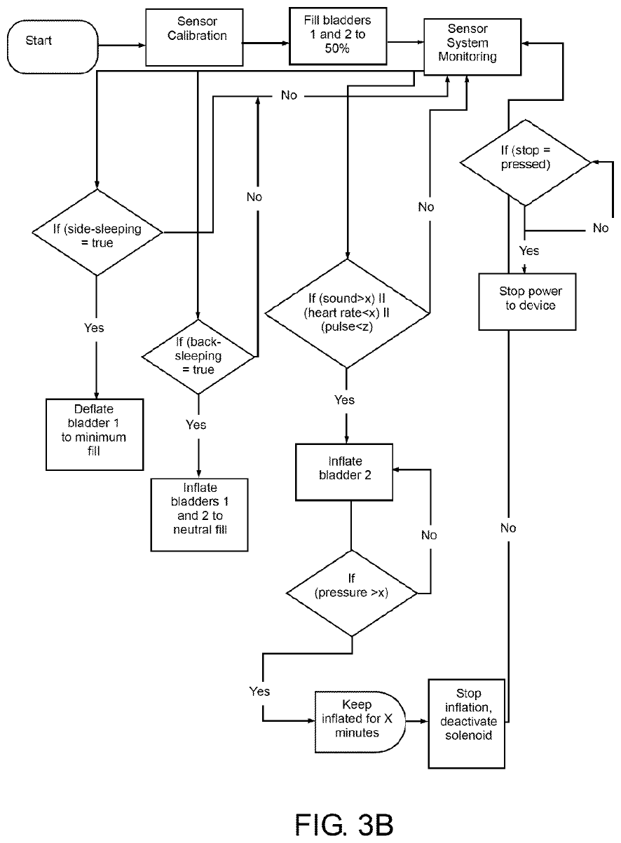

The number, position and shape of chambers 110 (or other actuators) of the adjustable positioning devices hereof may vary between different embodiments. The chambers/bellows may be positioned around the entire circumference of the neck or may extend around only one or more portions thereof. One or more actuators such as inflatable chambers or bellows positioned in the front portion of the neck, in the vicinity of the chin (to control chin life/angle of the mandible), are present in many embodiments hereof. FIG. 3A illustrates an embodiment of an adjustable positioning device 100a which includes a plurality of chambers 110a in the front portion of the neck (that is, in the area or vicinity of the chin) only. In the embodiment of FIG. 3A, three upper chambers 110a and three lower chambers 110a are provided in a stacked arrangement for a total of six chambers 110a. Support or collar 112a to which chambers 110a are attached extends around the back of the patient's neck. As illustrated in the rightmost view, positioning device 100a may include a soft outer cover 102a or shroud (formed, for example, from a soft cloth or other material) enclosing a plurality of inflatable volumes, chambers, bladders, or bellows 110c connected to support 112a.

In the embodiment of FIG. 3A, chambers 110a are in fluid connection with a manifold/distribution system of a dedicated control system 130a including electronic circuitry systems or components within a housing 130a′ and a sensor system 130a″ (for example, including one or more, pressure sensors, flow sensors, CO₂ sensors, sound sensors, etc. as described herein). As illustrated in the embodiment of FIG. 3A, the electronic circuitry includes n (for example, n=6 in the embodiment of FIG. 3A) electrically-powered air pumps 134a to independently control inflation of inflatable chambers 110a of positioning device 100a. The state of the air pumps is controlled via solenoids 132a which are in operative connection with a microprocessor 152. In a representative studied embodiment, the microprocessor is operatively connected to a printed circuit board 150 compatible with an ARDUINO system (available from Arduino of, for example, Somerville, MA USA) as known in the art. In a number of embodiments, pressure/flow rate sensors of sensor system 130a″ were placed in fluid connection with conduits or tubes 112a which placed air pumps 132a in fluid connection with inflatable chambers 110a. In some embodiments, all or part of the control system may be integrated into a positioning device hereof such as positioning device 110a. A data input/output and/or communication system 156a and/or other electronic circuitry components may be provided as described above and as known in the art.

A flowchart setting forth an embodiment of a process for operating a system hereof such as system 100a in the case of a three-volume, a three-chamber, three-bellows, or three-bladder system is illustrated in FIG. 3B via a control system such as control system 130a. The electronic circuitry of FIG. 3B is, for example, operable to control inflation of three chambers 110a. The control software may, for example, be programmed to control one chamber independently of the other two chambers, which are controlled together or in unison. Alternatively, all three chambers may be controlled independently. As, for example, illustrated in the embodiment of FIG. 3B, two or more of a plurality of chamber may be controlled together while one or more other chambers are controlled independently. For example, two side chambers, under the chin, may be controlled together while a back chamber is controlled independently thereof.

FIG. 4 illustrates another embodiment of an adjustable positioning device 110b hereof which is formed in the conformation of a neck pillow as commonly used in cars, planes etc. However, the orientation of positioning device 110b when worn by a user is reversed compared to the common orientation of a neck pillow. In the illustrated embodiment, four inflatable chambers 110b are positioned around positioning device 100b. Such chambers are connected integrally and form a portion of a support system or collar as described above. In the embodiment of FIG. 4, chamber or bellows 110b form a frontal (referring to the orientation of positioning device 100b when worn by a patient) portion of the collar and an optional connecting strap 114b is provided at the rear thereof to assist in maintaining positioning device 110b in operative connection with the patient's neck. Conduits 142b extend to a control system including, for example, a manifold or distribution system as described above.

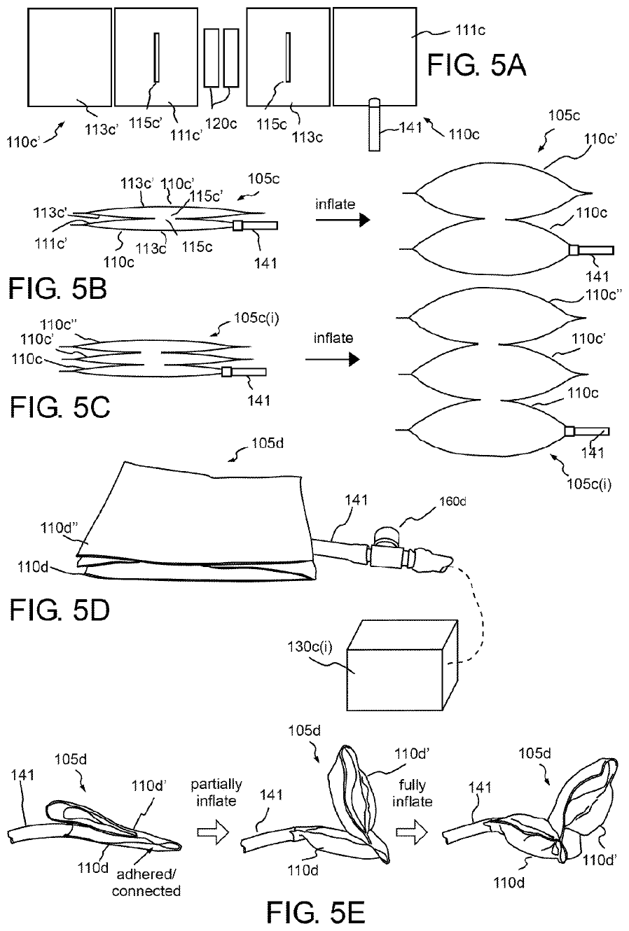

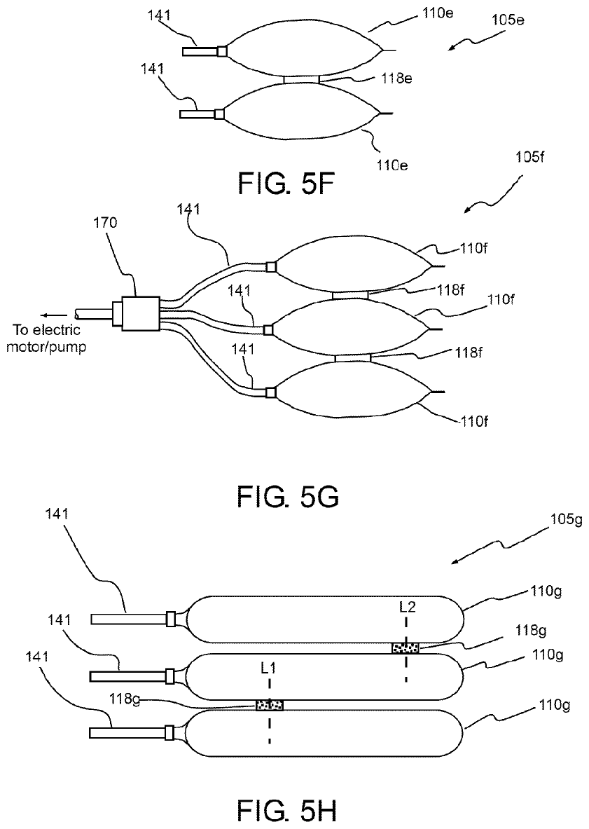

In a number of embodiments, an actuator hereof includes a plurality of inflatable chambers which are combined, forming a multi-chamber or composite-chamber pneumatic actuator. Such actuators may for example, include stacked chambers which are sometimes referred to herein as a bellows system. In a number of embodiments, each of the volumes, compartments or chambers of the bellows system is in fluid connection with one or more other volumes, compartments or chambers of the bellows system. FIG. 5A illustrates the components of a representative embodiment of a two-chamber or two-compartment actuator (in the form of a bellows system or stacked chamber system) hereof in a disassembled state. In the illustrated embodiment a first chamber 110c includes a connector 141 sealingly attached to a first (bottom) layer 111c over the length of the seal therewith. In forming first chamber 110c, a second (top) layer 115 is sealed to first layer 111c around the perimeters thereof, and also forms or completes a sealed connection with connector 141. Second layer 113c includes an opening or passage 115c formed therein. Section 120 of material may be connected to second layer 113c around passage 115c to help in forming a sealed connection between first chamber 110c and a second chamber 110c′. Second chamber 110c′ includes a first (bottom) layer 111c′ which includes a passage 115c′ formed therein. In forming second chamber 110c′, a second layer 113c is sealed to first layer 111c′ around the perimeters thereof. When first chamber 110c is sealed to second chamber 110c′ as illustrated in FIG. 5B, passages 115c and 115c′ align to form a fluid connection between first chamber 110c and second chamber 110c′. As clear to one skilled in the art, the method of forming stacked chambers in a bellows system as described above (and/or other methods) can be used to create a bellows system including three or more chambers. FIG. 5C illustrates a bellows system 105c(i) including three chambers 110c, 110c′ and 110c″. Any chamber that is positioned intermediate between two other chambers will include a passage formed in each of the first layer (or first side/position) and the second layer (or second side/position) thereof (as illustrated for chamber 110c′ in FIG. 5C) to form a fluid connection with each of the adjacent chambers (110c and 110c″ in FIG. 5C).

FIG. 5D illustrates a perspective view of an actuator including a two-chamber bellows system bereof which is in operative connection with a control unit 130c(i) therefor via a valve 160d. FIG. 5E illustrates perspective views of two-chamber bellows system 105d wherein the chambers 110d and 110d′ are adhered/connected on one side thereof to allow comparatively greater expansion on the other side. FIG. 5E illustrates bellows system 105d in a deflated, a partially inflated, and a fully inflated state. The manner of connecting chamber of a multi-chamber system hereof may thus control the manner of inflation/actuation. The manner in which a flexible/inflatable pneumatic actuator such as a chamber or chamber system hereof inflates may also be controlled by selection of materials therefor. For example, one side of a chamber may be formed from a material that is less flexible or more rigid than the remainder of the chamber. Bellows system 105d and/or other bellows systems hereof may, for example, be used as an actuator to effect positioning such as chin/head tilt without expanding into or toward a patient's neck/throat (which may cause discomfort) as illustrated, for example, in FIG. 9 discussed below.

In the embodiment of FIGS. 5A through 5E, a single air/gas line is used to fill multiple chambers of an actuator including a bellows system or a stacked chamber system hereof wherein each of the chambers are in fluid connection with each other. FIGS. 5F through 5H illustrate embodiments of pneumatic actuators hereof wherein a bellows system or stacked chamber systems includes two or more connected chambers which are not in direct fluid connection with each other. A separate gas/air connection line is placed in fluid connection with each such chamber. FIG. 5F illustrates an embodiment of an actuator hereof including a two-chamber bellows system or stacked chamber system 105e wherein each chamber 110e has an independent air supply line or connector line 141 in fluid connection therewith, and each chamber 110e is attached to the adjacent chamber 110e (for example, via an adhesive 118e). FIG. 5G illustrates a side view of an actuator hereof including a three-chamber bellows system 105f wherein each chamber has an independent air supply line or connector line 141, and each chamber 110e is attached to the adjacent chamber(s) (for example, via an adhesive 118e). Each of connector lines 141 in FIG. 5G is attached to a 4-way connector valve 170. 4-way connector 170 may be a passive connector which merely splits flow between the chambers evenly or 4-way connector 170 may be an actively controlled valve to control the flow to the connected chambers independently. FIG. 5H illustrates a side view of an actuator hereof including a three-chamber bellows system 105g wherein each chamber 110g has an independent air supply line or connector line 141, and each chamber 110g is attached to the adjacent chamber(s) 110g (for example, via an adhesive 118g) on different axes of attachment represented by broken lines L1 and L2. Offsetting connections between compartments or chambers 110g coupled with independent chamber control may, for example, provide an alternative route for adjusting control or motion/positioning in multiple degrees of freedom for a pneumatic actuator hereof. In comparing chambers 110f to 110g (see FIGS. 5G and 5H, respectively), it is seen that the shape of the individual chambers or other inflatable systems hereof is readily changed to, for example, provide smooth edges and/or to accommodate a certain use. For example, inflatable system and/or other actuators hereof may be shaped and dimensioned to fit into a particular positioning device, for patient comfort, to achieve a desire controlled motion thereof, etc. Inflatable systems hereof may, for example, be oversized to create a pillow-like sensation upon inflation.

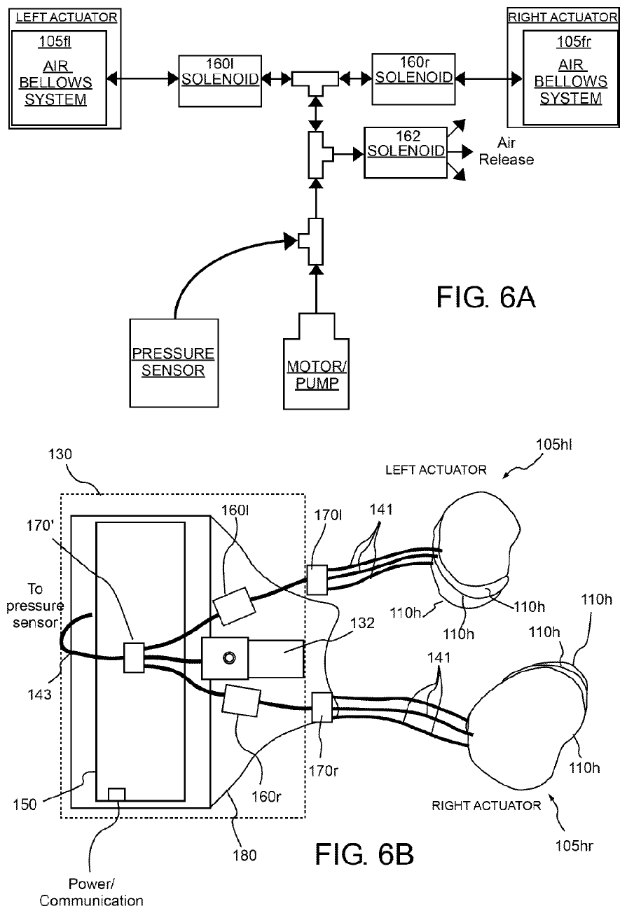

FIG. 6A illustrates a block diagram of air flow through an embodiment of a system hereof including two actuators (a right actuator and a left actuator) wherein each actuator includes a bellows systems or stacked chambers systems as, for example, illustrated in FIGS. 5B through 5G. In the embodiment of FIG. 6A, flow through the system is controlled by solenoid actuated valves. In the illustrated embodiment, flow to and from a left air bellows system 105fl is controlled via solenoid actuated valve 160l, and flow to and from a right air bellows system 105fr is controlled via solenoid actuated valve 160r. In the illustrated embodiment, a solenoid actuated valve 162 is provided to release air from the system. In a number of embodiments, all solenoid actuated valves 160l, 160r, 162 are normally open (that is, under no-power scenarios, all solenoid actuated valves are open). This mode of operation provides pressure release in case of failure/loss of power. A pressure sensor as described herein may also be in fluid connection with the system.

FIG. 6B illustrates a top view of another representative embodiment of a system hereof including two actuators wherein each actuator includes an air bellows systems or stacked chambers systems 105hr and 105hl which are positioned on a right side and a left side of a person/patient, respectively, during use of a positioning device hereof as described further below. Each of bellows systems 105hr and 105hl includes three chambers 110b, which are connected to four-way connector valves 170r and 1701 via connector lines/tubing 141 and thereby to solenoid valves 160r and 160l, respectively, of a control system 130. Solenoid valves 160l and 160r. as well as motor/pump 132, are connected to a four-way connector 170′. A connector, tube or conduit 143 connects four-way connector 170′ to a pressure sensor (not shown) on a board 150 such as a printed circuit board as known in the art (and as described above in connection with FIG. 3A) In the embodiment of FIG. 6B, flow through the system is controlled by the solenoid actuated valves which are in communicative connection with a processor system of control system 130. Flow to left air bellows system 105hl is controlled via solenoid actuated valve 160l, and flow to right air bellows system 105hr is controlled via solenoid actuated valve 160r. In the illustrated embodiment, various elements of control system 130 are attached to a base 180.

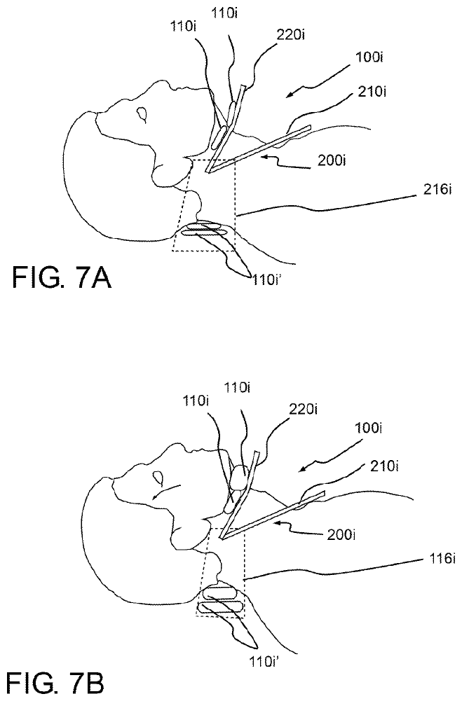

In a number of embodiments of adjustable positioning device or systems hereof, the wearable support used to place the device or system in operative connection with a person or patient includes a wearable frame. FIGS. 7A and 7B illustrate a system 100i including wearable frame 200i. Frame 200i (which is illustrated schematically in FIGS. 7A and 7B) includes a lower, base, or sternum/chest section 210i which rests on the patient sternum/chest. Frames hereof may partially or fully encompass the neck of the user. Frame 200i further includes an upper or support section 220i which supports one or more actuators including inflatable chambers 110i (and/or one or more bellows systems as described herein) to interact with and position the patient's head/chin. In that regard, chambers 110i are inflatable to contact a person's head (for example, the chin/jaw region—that is, tissue at the base of the mandible) to position the head/neck as described herein. FIG. 7B illustrates chambers 110i inflated to position the head, chin and neck as described herein. Device or system 100i (or other devices/systems hereof) may also include an actuator or actuators including chambers 110′ positioned at the posterior cervical neck to increase the chin lift angle upon inflation thereof. The lower surface of lower frame section 210i may be padded (for example, using a foam material such as a polymeric foam as known in the cushioning arts) to increase comfort of the user. Frame 200i may, for example, be designed in certain respect to be similar to a neck brace as is known in the medical arts. A rear collar section 216i may be provide for support of frame 200i around the neck of the user and to incorporate rear compartments 110i′.

In a number of the embodiments described above, a portion of the surface of the actuator, in the form of an inflatable chamber or multi-chamber system, functions as a contact member to contact the patient's head and impart motion thereto in positioning the head. Interface or contact members hereof may alternatively be formed as elements or components separate from and connectible to the actuator(s) hereof. For example, in a number of representative embodiments in which adjustable positioning device or systems hereof include a wearable frame, a upper or support section of the frame may include or function as a moveable contact member. For example, at least a portion of an upper section of the frame may be moveable relative to the lower, base or sternum/chest section of the frame. The upper section of the frame (or a portion thereof) may be brought into contact with the patient's head (for example, the chin/jaw—that is, tissue at the base of the mandible) to position the head/neck as described herein. Once or more actuators, which may, for example, include inflatable chambers and/or bellows systems as described herein, may be provided in connection with the upper section to control the position of the upper section (of portions thereof which function as movable contact members) relative to the lower section and thereby position the head/neck of the patient.

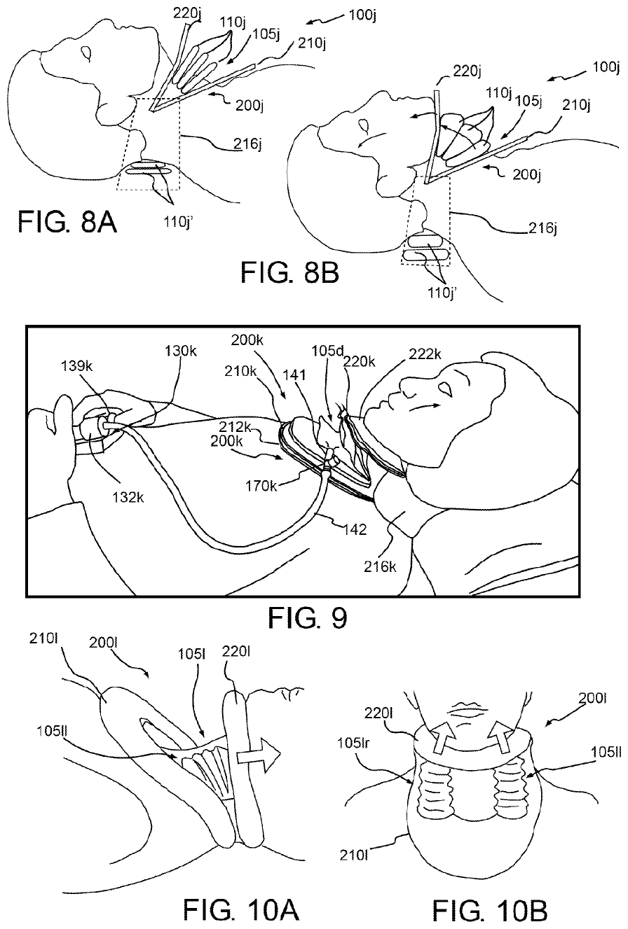

FIGS. 8A and 8B illustrate a system 100j including a wearable frame 200j that is somewhat similar to frame 200i. Frame 200j (which is illustrated schematically in FIGS. 8A and 8B) includes a lower, base, or stemum section 210) which rests on the patient sternum/chest. Frame 200j further includes an upper or contact section 220j which is designed to move (for example, pivot) relative to lower section 210j to contact and place force upon the chin, jaw, or lower mandible region of the patient to interact with and position the patients head, chin and neck as described herein. In that regard, an actuator including a bellows system 105j of system 100j includes chambers 110j (three, in the illustrated embodiment) which are inflatable to move upper section 220j as illustrated in FIG. 8B to contact a person's head (for example, the chin/jaw—that is, tissue at the base of the mandible) and thereby position the head/neck as described herein. As described in connection with device or system 100i, system 100j may further include one or more actuator such as chambers 110j′ positioned at the posterior cervical neck to increase the chin lift angle upon inflation thereof. The lower surface of lower (frame) section 210j as well as the upper surface of upper (frame) section 220j may be padded (via passive padding and/or via one or more inflatable chambers hereof) to increase comfort of the user. System 100j may further include a collar or neck strap section 216j connected to frame 200j (for example, for additional support of frame 200j) which extends around the back of the neck of the user. Collar or neck strap 216j may encompass or incorporate rear compartments 110j′. Frame 200j may, additionally or alternatively, be horseshoe shaped to partially encompass the back of the patient neck (for example, similar to the shape of positioning device 100b of FIG. 4) to maintain frame 200j in operative connection with the patient's neck.

FIG. 9 illustrates another embodiment of a system 100k hereof including a wearable frame 200k. Frame 200k includes a lower (frame) section 210k and an upper (frame) section 220k, which is moveable (for example, pivotable) relative to lower section 210k as described above in connection with frame 200j. Padding sections 222k and 212k are provided on the upper surface of upper section 210k and the lower surface of lower section 210k, respectively, to increase the comfort of the user. The position of upper section 220k relative to lower section 210k is controlled by the state of a pneumatic actuator including bellow system 105d as described in connection with FIG. 5D. An actuator including a bellows system 105d is placed in fluid connection with a motor/pump 132k of a control system 130k via connector tubing 141, extending tubing 142 and an intervening valve 170k to control the state of inflation of bellow system 105d. In illustrated system 100k, control system 130k consists of or includes a handheld controller. Manual control of the state of inflation of bellow system 105d (and thereby the position of head, chin and neck) may be, at least partially, controlled via interaction with a manual control member 139k.

In the embodiment of FIGS. 10A and 10B, the position of upper section 220l of frame 200l relative to lower section 210l thereof is controlled by a left actuator and a right actuator, each of which includes a bellows sections 105lr and 105ll, respectively, which are positioned on the right side and left side of frame 200l (with respect to the patient when wearing frame 200l). The state of each actuator (that is, the state of inflation of each of bellows systems 105lr and 105ll) may, for example, be independently controlled via a control system such as illustrated in FIGS. 6A and 6B.

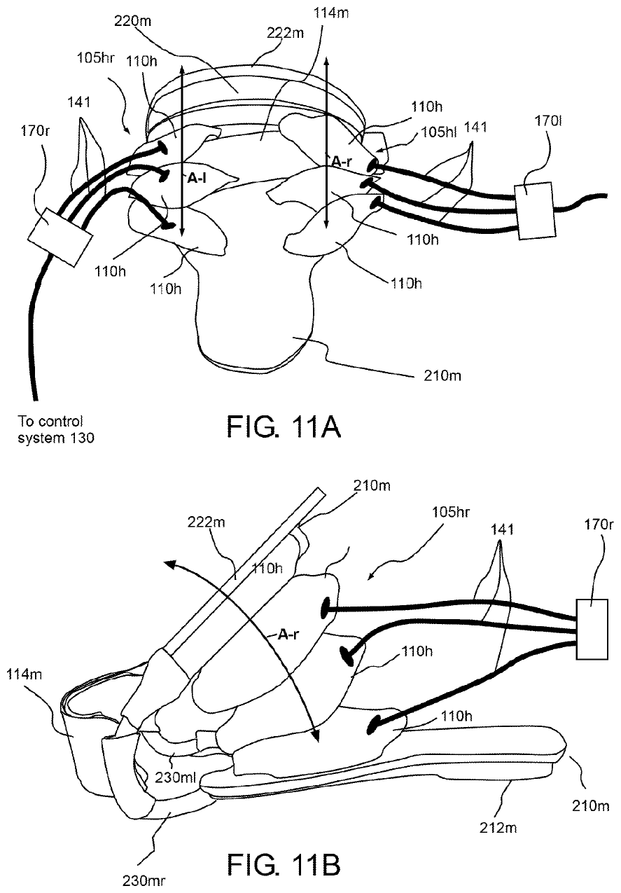

FIGS. 11A and 11B illustrate another embodiment of a wearable frame 200m hereof. The position of upper section 220m of frame 200m relative to lower section 210m thereof is controlled by two actuators in the form of bellows systems 105hr and 105hl (as illustrated in FIG. 6B) positioned on the right side and left side of frame 200m, respectively. Bellows systems 105hr and 105hl may be independently controlled by control system 130 as described in connection with FIG. 6B. In the embodiment of FIGS. 11A and 11B, upper section 220m and lower section 210m are connected by semi-rigid or flexible and resilient members 230mr and 230ml (for example, formed from a flexible or semi-rigid metal such as steel). Resilient members 230mr and 230ml may be removably connected to lower section 210m and upper section 220m. One may, for example, choose resilient members that are sized and/or otherwise designed for a particular patient. Likewise, one may choose a lower section and/or an upper section that is/are sized and/or otherwise designed for a particular patient. Each of the actuators (bellows systems 105hr and 105hl) abuts (and is typically connected to) a lower surface of upper section 220m at a top thereof and abuts (and is typically connected to) an upper surface of lower section 210m at a bottom thereof. The number of actuator(s) hereof and other aspects (for example, size, shape, etc.) may likewise be chosen for use with a particular patient. Such actuators may be removably attachable to the frame to facilitate a modular design. Removable connections of various elements of positioning devices hereof also facilitate replacement of defective components.

Via control of the actuators (that is, controlled inflation/deflation or pressurization of compartments/chambers 110h of right bellow systems 105hr and left bellows system 105hl), the amount of flex in right (or right side) resilient member 230mr and left (or left side) resilient member 230ml, respectively is independently controlled. A clam-shell-like motion of frame 200m is thereby created (wherein upper section 220m may be tilted or angled with respect to lower section 210m) as represented by independent arrows A-l and A-r in FIGS. 11A and 11B. Resilient members 230mr and 230ml operate as resilient or flexible hinges, and return frame 200m and device 100m to an inactivated or neutral state upon depressurization/deflation of chambers 110h of bellow systems 105hr and 105hl. Resilient members or hinges 230mr and 230ml may also function as connection points for an optional adjustable neck strap or partial collar 114m.

In FIG. 11B, the left-side actuator (bellows system 105hl) is absent or removed for clarity. As described above, an upper cushioning or padding member 222m and a lower cushioning or padding member 212m may be attached to an upper side of upper section 220m and a lower side of lower section 210m, respectively, to improve patient comfort.

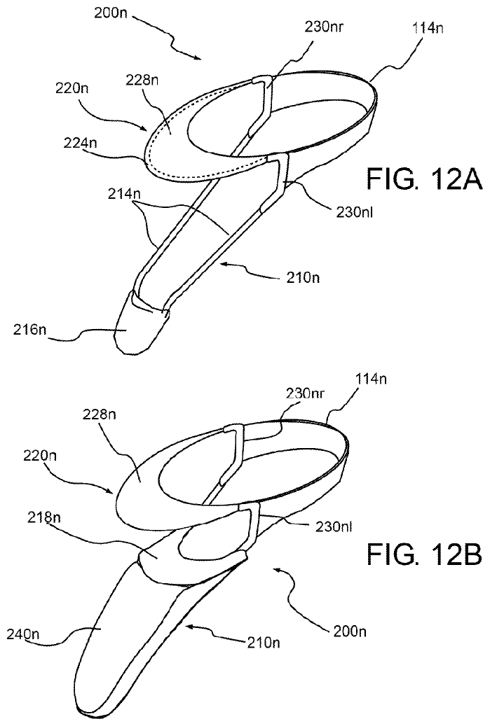

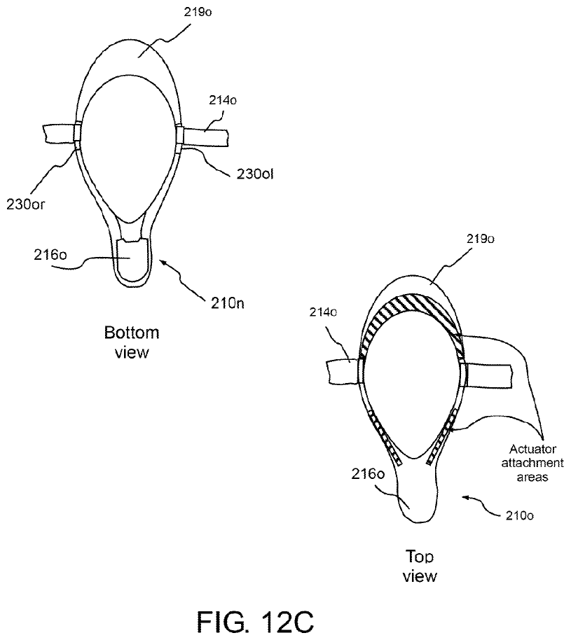

FIGS. 12A and 12B illustrate a further embodiment of a support including a frame 200n hereof without any actuator (for example, bellows systems and/or other motion-imparting systems) installed therein. As discussed in connection with frame 200m, flexible and resilient members or hinges 230nr and 230nl of frame 200n operate independently as flexible hinges via control of the pressurization of right and left bellows systems (not shown). Resilient members or hinges 230nr and 230nl connect to upper section 220n and lower section 210n to allow for independent, isolated right and left side movement. Resilient members 230nr and 230nl may also operate as connection points for an optional adjustable neck strap or collar 114n as described in connection with frame 200m. Neck strap 114n may be soft and/or cushioned/padded to comfortably secure the device in operative connection with the user.

Lower (frame) section 210n rests against the user's chest and provides a stable support for one or more actuators hereof. In a number of embodiments, lower section 210n of frame 200n is fabricated using coated, rigid, extending wire or rod element 214n (for example, formed from steel) to provide a low profile. As illustrated in FIG. 12A, a contact member 216n, which may be padded, is attached to wire/rod frame element 214n at a distal end. In a number of embodiments, upper (frame) section 220n was formed from a coated, padded, and rigid wire frame element 224n (for example, formed from steel) which rests below the user's jawline. An upper platform section 228n may be provided in connection with rigid wire/rod frame element 224n to interact with, for example, left and right actuator systems as described above.

Lower section 210n and upper section 220n (and other frame elements hereof) need be sufficiently rigid in one or more areas thereof to provide support for actuators such as inflatable chamber or bellow system hereof so that head positioning as described herein is achievable. At least a portion or portions of the lower section 210n and upper section 220n may be sufficiently flexible, moveable, and/or adjustable to provide user comfort.

As illustrated in FIG. 12B, a housing 240n and a base or platform 218n (which may be connected or form integrally with housing 240n) may be attached to frame element 224n. Housing 240n may be curved and padded and/or somewhat flexible to increase user comfort and reduce, minimize, or eliminate the risk of injury. Housing 240n may, for example, be used to enclose all or a portion of the electrical circuitry of a system hereof. Housing may, for example, be shaped to create a small or minimal footprint on the chest. Lower frame platform 218 provides a base for attachment of actuator(s) hereof.

FIG. 12C illustrates a top view and a bottom view of another embodiment of a lower section of an embodiment of a frame hereof. The frame includes a lower or bottom section 210o (including a forward contact section 216o) and an upper section (not shown) which may be similar to upper section 220n. In FIG. 12C, lower section 210o is illustrated without actuators (for example, bellows systems and/or other motion-imparting devices) installed therein or attached thereto but actuator attachment areas are illustrated in the top view (right side). As discussed in connection with frames 200m and 200n, flexible and resilient members or hinges 230or and 230ol of lower section 210o of frame 200o operate independently as flexible hinges via control of the pressurization of right and left actuators (for example, bellows systems; not shown). Resilient members or hinges 230or and 230ol connect to the upper section and provide independent, isolated right and left side movement. Resilient members 230nr and 230nl may also operate as connection points for an optional adjustable neck strap or collar 114n as described in connection with frames 200m and 200n. In the embodiment of FIG. 12C lower section 210o also includes a rear section or platform 219o for attachment of one or more actuators such as bellow systems (not shown), which are operable to adjust neck position as described above (for example, in connection with FIGS. 7A and 7B).

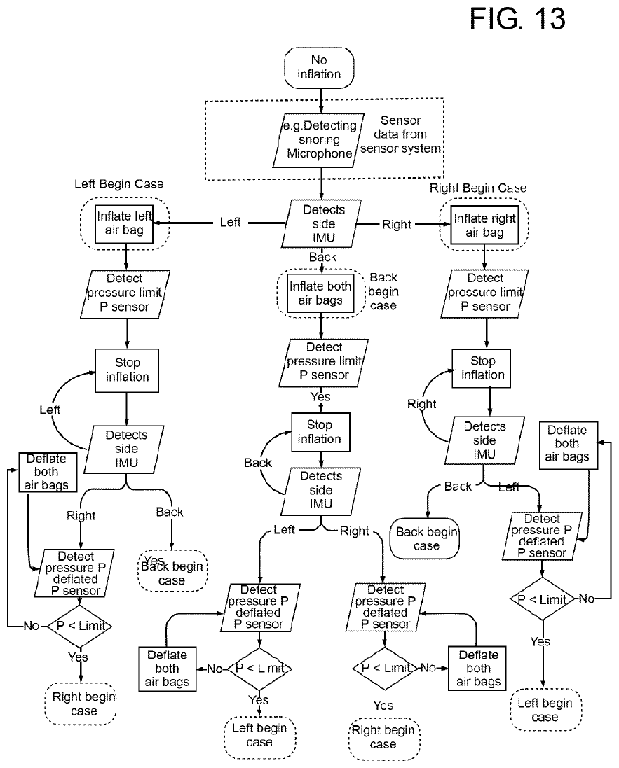

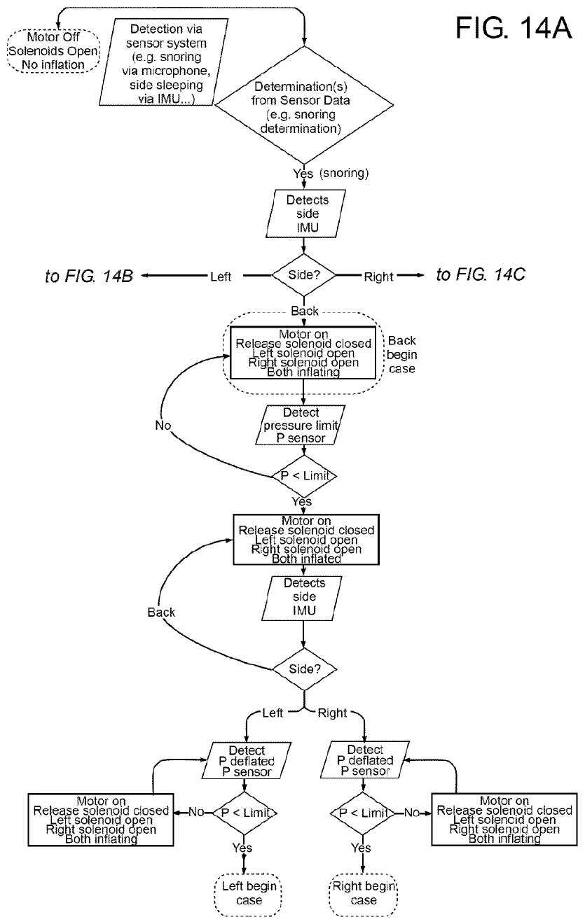

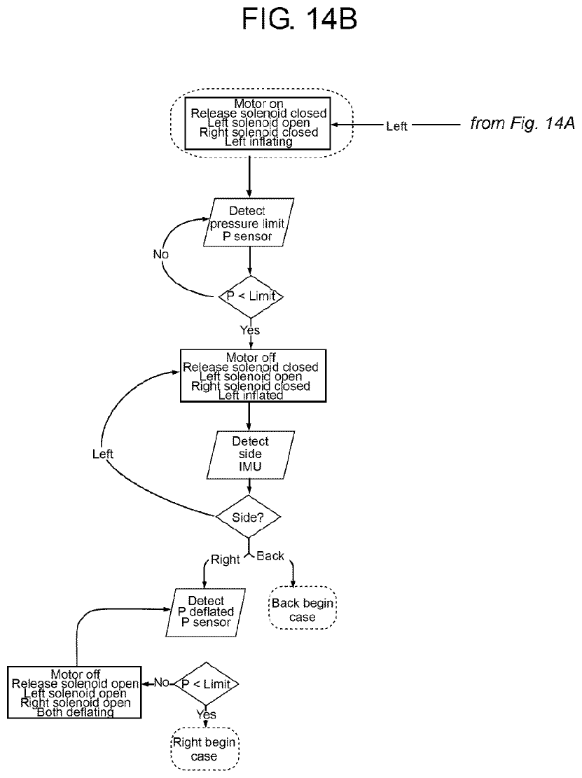

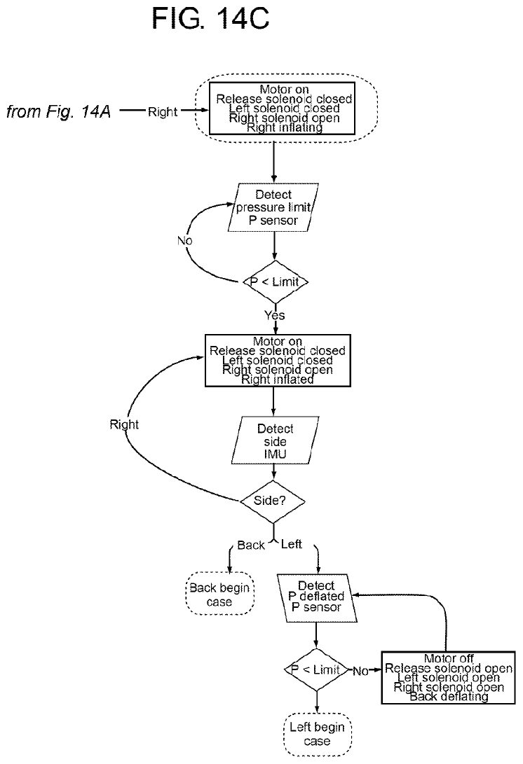

FIG. 13 illustrates an embodiment of a generalized flow chart for operation of a device or system hereof in which data from a sensor system hereof is used to determine, for example, the presence of snoring (using, for example, a microphone). Moreover, a sleeping position on the left/right side or back of the user may also be determined (for example, via IMU). Further, pressure (P) may be determined via a pressure sensor and, for example, compared to established pressure limit(s). A system hereof may be controlled, at least partially, on the basis of such data. FIGS. 14A through 14C illustrates an embodiment of a more detailed operational flow chart for a device or system hereof in which sensor system data is used to determine, for example, snoring and a sleeping position (for example, on the left/right side of back of the user). FIG. 14A provides a representative embodiment of an algorithm for the determination of sleep position and for control/operation after determination of a sleep on back position. FIG. 14B illustrates a portion of the operational flow chart setting forth an algorithm for control after determination of a left-side sleep position. FIG. 14C illustrates another portion of the operational flow chart setting forth an algorithm for control after a determination of a right-side sleeping position.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A system for use in connection with a patient, comprising:
a positioning device comprising one or more movable contact members to contact the patient, one or more actuators in connection with the one or more movable contact members, and a support to which each of the one or more actuators and each of the one or more contact members are operatively connected, the support being configured to be placed in operatively connection with the patient;
at least one source of energy in connection with the one or more actuators; and
a control system in operative connection with the one or more actuators and with the at least one source of energy to control a state of the one or more actuators via control of energy provided thereto to control a position of the one or more movable contact members and thereby control a position of the patient a determined manner.

2. The system of claim 1 wherein the position of the patient includes the position of at least one of the patient's head, chin, or neck.

3. (canceled)

4. The system of claim 2 wherein the control system comprises a processor system, a memory system in operative connection with the processor system, and at least one algorithm stored in the memory system and executable by the processor system to control the state of the one or more movable contact members.

5. (canceled)

6. The system of claim 4 further comprising a sensor system comprising one or more sensors in operative connection with the control system, the control system being configured to control the state at least one of the one or more actuators at least in part on the basis of data from the sensor system.

7. The system of claim 6 wherein the sensor system comprises at least one of a sound sensor, an oximeter sensor, a carbon dioxide sensor, a position sensor, a breath flow sensor, accelerometer, inclinometer, tilt sensor or a heart rate sensor.

8. (canceled)

9. The system of claim 6 wherein the control system is configured to determine at least one of a state of the patient's breathing and a position of the patient from the data from the sensor system and control at least one of the one or more actuators at least in part on the basis of at least one of the determined state of the patient's breathing and the position of the patient.

10. The system of claim 6 wherein the at least one algorithm is configured to store a maximum state and a minimum state for at least one of the one or more actuators.

11. (canceled)

12. The system of claim 6 wherein the at least one algorithm is configured to implement feedback control of the state of the at least one of the one or more actuators on the basis of data from the sensor system.

13. The system of claim 12 wherein the control system comprises at least one machine learning algorithm stored in the memory system and executable by the processor system to alter control of the state of the at least one of the one or more actuators on the basis of data from the sensor system measured over time.

14. The system of claim 13 wherein the sensor system includes a sound sensor and the machine learning algorithm is configured to differentiate at least one of snoring from other sounds and between sounds coming from more than one person.

15. (canceled)

16. (canceled)

17. The system of claim 2 wherein at least one of the one or more contact members is positioned to be placed in contact with at least one of a lower portion of the jaw line of the patient and the neck of the patient.

18. (canceled)

19. The system of claim 4 wherein the support comprises a frame comprising a lower section configured to contact a chest region of the patient and an upper section comprising one or more of the one or more movable contact members, the upper section being movable relative to the lower section, wherein at least one of the one or more actuators is in connection with the upper section and with the lower section to move the upper section relative to the lower section.

20. (canceled)

21. The system of claim 19 wherein the upper section is connected to the lower section by a first resilient member on a first side of the frame and by a second resilient member on a second side of the frame.

22. (canceled)

23. The system of claim 21 comprising a first actuator on the first side of the frame which is in connection with the upper section and with the lower section and a second actuator on the second side of the frame which is in connection with the upper section and with the lower section, the control system being configured to independently control the first actuator and the second actuator and thereby independently control the amount of flex in each of the first resilient member and the second resilient member.

24. The system of claim 1 wherein the source of energy comprises a pneumatic source of energy, the one or more actuators comprising an inflatable system in fluid connection with the pneumatic source of energy and the state of the at least one of the one or more actuators controlled by the control system is a state of inflation.

25. (canceled)

26. The system of claim 24 comprising a plurality actuators, each of the plurality of actuators comprising an inflatable system, wherein the control system is configured to control the state of inflation of at least one of the inflatable systems independently of the control of at least one other of the plurality of inflatable systems.

27. (canceled)

28. The system of claim 24 wherein the inflatable system comprises a plurality of connected inflatable chambers, each of the plurality of connected inflatable chambers being formed at least partially of a flexible material.

29. (canceled)

30. (canceled)

31. (canceled)

32. The system of claim 28 wherein two or more of the plurality of connected inflatable chambers are connected in a manner to control inflation of the inflatable system.

33. (canceled)

34. (canceled)

35. (canceled)

36. (canceled)

37. The system of claim 28 wherein two or more of the plurality of actuators are positioned at different circumferential positions on the support, wherein the support extends around at least a portion of the circumference of the patient's neck when the positioning device is placed in operative connection with the patient's neck.

38. A positioning device for use in connection with a patient, comprising: one or more movable contact members to contact the patient, one or more actuators in connection with the one or more movable contact members, and a support to which each of the one or more actuators and each of the one or more contact members are operatively connected, the support being configured to be placed in operative connection with the patient, each of the one or more actuators being adapted to be placed in connection with at least one source energy, wherein a state of the one or more actuators is controllable via control of energy provided thereto from the at least one source of energy to control a position of the oner or more movable contact members and thereby control a position of the patient in a determined manner.

39. (canceled)

40. (canceled)

41. A method of positioning a patient, comprising:
providing a system which comprises
a positioning device comprising one or more movable contact members to contact the patient, one or more actuators in connection with the one or more movable contact members, and a support to which each of the one or more actuators and each of the one or more contact members are operatively connected, the support being configured to be placed in operatively connection with the patient;
at least one source of energy in connection with the one or more actuators; and
a control system in operative connection with the one or more actuators and with the at least one source of energy to control a state of the one or more actuators via control of energy provided thereto to control a position of the one or more movable contact members and thereby control a position of the patient in a determined manner. placing the positioning device in operative connection with the patient; and
operating the control system to control a position of the one or more movable contact members and thereby control the position of the patient.

42.-51. (canceled)
